(19) United States
(12) Patent Application Publication
(10) Pub. No.: US 2026/0054092 A1
(43) Pub. Date: Feb. 26, 2026

(54) BALLOON DEVICES FOR DELIVERING LIGHT THERAPY AND METHODS FOR USING THEM

(71) Applicant: CERN CORP., Trabuco Canyon, CA (US)

(72) Inventor: Gregg A. Klang, Coto de Caza, CA (US)

(21) Appl. No.: 17/001,609

(22) Filed: Aug. 24, 2020

Related U.S. Application Data
(60) Provisional application No. 62/890,183, filed on Aug. 22, 2019.
(60) Provisional application No. 63/012,708, filed on Apr. 20, 2020.

(51) Int. Cl. A61N 5/06

(52) U.S. Cl. CPC A61N 5/0624; A61N 2005/0611; A61N 2005/0626; A61N 2005/063; A61N 2005/0635; A61N 2005/0651; A61N 2005/0664

(57) ABSTRACT

Devices, systems, and methods are provided for vaginal light therapy of a subject using a device carrying an expandable member and one or more light sources. A distal end of the device carrying the expandable member thereon is introduced a vaginal canal with the expandable member in a contracted condition, the expandable member is expanded within the vaginal canal, the one or more light sources are activated to emit light outwardly at one or more wavelengths within a range of germicidal or other therapeutic light; and the device is removed from the vaginal canal after treatment.

### RELATED APPLICATION DATA

The present application claims benefit of co-pending U.S. provisional applications Serial Nos. 62/890,183, filed Aug. 22, 2019, and 63/012,708, filed Apr. 20, 2020, the entire disclosures of which are expressly incorporated by reference herein.

### FIELD OF THE INVENTION

The present application generally relates to medical devices and, more particularly, to vaginal light therapy devices, e.g., for treatment of bacterial and fungal infections, and to systems and methods for using such devices.

### BACKGROUND

Vaginitis is characterized by inflammation of the vagina that results in discharge, itching and pain. The cause is usually due to a change in the normal balance of vaginal bacteria or an infection. As many as 75% of the female population experience bacterial or fungal infection within their vagina at least once during their lifetime. The symptoms range from mucus-like discharge, itching, aching, pain during intercourse to odor. The vaginal infections often have multiple causes that present challenging cases for treatment.

It is critical to have a balance between naturally occurring yeast and bacteria. It is when the system is out of balance or other types of bacteria or yeast are present within the environment does one end up with vaginitis. Sometimes the reduction in good bacteria due to use of antibiotic allows for a propagation of yeast, typically Candida albicans resulting in yeast infection. Further, either a change in pH balance or introduction of foreign bacteria in the vagina leads to infectious vaginitis. Physical factors that contribute to the development of an infection include the following: constantly wet vulva due to tight clothing, chemicals coming in contact with the vagina via scented tampons, antibiotics, birth control pills, or a diet favoring refined sugar and yeast.

Bacterial vaginosis also known as vaginal bacteriosis or Gardnerella Vaginitis is a disease of the vagina caused by excessive bacteria growth. Common symptoms include increased vaginal discharge that often smells fishlike. The discharge is usually white or gray in color. Burning with urination may also occur. Itching is uncommon. Occasionally there may be no symptoms. Having bacterial vaginosis increases the risk of infection by a number of other sexually transmitted infections including HIV/AIDS. It also increases the risk of early delivery among pregnant women. Bacterial vaginosis is caused by an imbalance of the naturally occurring bacteria in the vagina. Diagnosis is suspected based on the symptoms and may be verified by testing the vaginal discharge and finding a higher than normal vaginal pH and large numbers of bacteria.

Bacterial vaginosis is often confused with a vaginal yeast infection. Usually treatment is through the use of antibiotics. Bacterial vaginosis is the most common vaginal infection in women of reproductive age. The percentage of women affected at any given time varies but can be as high as 35%. Antibiotics, administered either orally (e.g., metronidazole) or vaginally (e.g., clindamycin, metronidazole) are effective in treatment. About 10% to 15% of people, however, do not improve with the first course of antibiotics and recurrence rates of up to 80% have been documented. Recurrence rates are increased with sexual activity with the same pre-post treatment partner and inconsistent condom use although estrogen-containing contraceptives decrease recurrence. There is evidence of an association between bacterial vaginosis and increased rates of sexually transmitted infections such as HIV/AIDS. Bacterial vaginosis is associated with up to a six-fold increase of HIV shedding. There is also a correlation between the absence of vaginal lactobacilli and infection of Neisseria gonorrhoeae and Chlamydia trachomatis. Bacterial vaginosis is a risk factor for viral shedding and herpes virus type-2 infection. Bacterial vaginosis may increase the risk infection or reactivation of HPV.

Candidiasis, more commonly referred to as a yeast infection, is most commonly caused by an overgrowth of a fungus called Candida albicans in the vagina. Candida is yeast, a type of fungus. Yeast is usually present in the vagina in small numbers, and symptoms only appear with overgrowth. Candida can multiply when an imbalance occurs, such as when the normal acidity of the vagina changes or when hormonal balance changes. Frequently occurring yeast infections may be a sign of more serious overarching health problem such as diabetes or a compromised immune system. Recurrent infections may also be due to use of antibiotic medications. About 5-8% of women experience four or more episodes per year, diagnosed as recurrent vulvovaginal candidiasis. About 75% of all premenopausal women develop thrush at some point in their lives. With the introduction of over-the-counter medications for home treatment of yeast infections, many women elect to self-diagnose and self-medicate, indicating that the true incidence of yeast infections annually may be significantly under reported.

In comparison to antibacterial therapy, antifungal treatment is limited to a very small number of drug substances. Treatment for fungal infection can be topical or systemic. Topical antifungals, such as miconazole (over the counter Monistat), are generally considered as first-line therapy for uncomplicated, superficial, relatively localized fungal infections due to their high efficacy and low potential for systemic adverse effects. Systemic antifungal agents are absorbed and delivered to the body through the blood stream. The oral route is usually the safest, the most economical, and the easiest route for systemic antifungal drugs. Oral fluconazole is available by prescription.

Topical antifungal creams and suppositories have fewer side effects than oral antifungal medications because they aren't absorbed as readily, systemically by the body, and only exert a localized effect on the genital region. Antifungal pills affect the entire body, and side effects can include nausea, headaches, and abdominal pain. However, topical medications can be messy and uncomfortable, while pills are comparatively simple. Treatment using antifungal medication is ineffective in up to 20% of cases. Treatment for thrush is considered to have failed if the symptoms do not clear within 7-14 days. In addition, the incidence of resistance to antifungal agents may be increasing, with drug-resistant fungal strains becoming increasingly common causes of infection in high-risk patient groups such as HIV/AIDS patients. Accordingly, alternative antifungal strategies are being actively sought.

Severe forms of infection are hard to treat, and frequently require more aggressive and long-term therapy, as is the case with chronic, recurrent cases. Additionally, incomplete treatments often result in drug resistant infections therefore full course of therapy should be adhered to.

Although light therapy treatment of various bacterial, fungal or viral infection is generally known, a treatment of such infections is generally achieved through chemical or drug therapies. A use of such therapies affects an internal functioning of the vagina and uterus as the chemicals used in the form of paste or gel or liquid result in unwanted chemical reactions that are harsh or result in various complications.

Oral antifungal medications carry the risk of significant side effects, and many patients are allergic to or intolerant of these drugs. Topical solutions can be messy and inconvenient. There are no existing products for the treatment of yeast infections without also requiring medication. Hence there is a need for a product that allows for the treatment of yeast and bacterial infections quickly and simply without systemic effects. With the continued and accelerating emergence of antibiotic-resistant microorganisms, there is burgeoning interest and investment in light therapy. A device that leverages this rising technology could potentially gain rapid acceptance in specific use cases as well as broader support among the general population simply wishing to avoid exposure to additional medications.

In the view of the foregoing, improved devices and methods for treating intravaginal infections and/or other conditions would be useful.

### SUMMARY

The present application is directed to medical devices and, more particularly, to vaginal light therapy devices, e.g., for treating bacterial and fungal infections, such as vaginitis, for treating pelvic inflammatory diseases, and/or for performing vaginal rejuvenation therapy, and to systems and methods for using such devices.

In accordance with an exemplary embodiment, a light treatment device is provided that includes an elongate member including a proximal end, a distal end sized for introduction into a vaginal canal, one or more light sources on the distal end, and an expandable member carried on the distal end and surrounding the one or more light sources. The expandable member may be configured to emit light outwardly from the one or more light sources at one or more wavelengths, e.g., within a range of germicidal light. Optionally, the device may also include an inflator for expanding the expandable member from a contracted or delivery condition for introduction into a vaginal canal, and an expandable condition once inserted for delivering the light to treat one or more conditions.

In accordance with another embodiment, a method is provided for vaginal light therapy of a patient that includes inserting a distal end of an elongate light treatment device carrying an expandable member thereon into a vaginal canal with the expandable member in a contracted or delivery condition; expanding the expandable member within the vaginal canal; activating one or more light sources to emit light outwardly at one or more wavelengths, e.g., within a range of germicidal or other therapeutic light; and removing the device from the vaginal canal after treatment. For example, the device may be used to deliver low-level germicidal light, e.g., to treat targeted pathogens, for example, to treat fungal or bacterial vaginitis. Alternatively, the device may be used to treat other conditions, such as pelvic inflammatory disease, and/or for performing vaginal rejuvenation therapy.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures:

FIG. 1 shows an exemplary embodiment of a device for vaginal light therapy of a patient including an expandable member carried on a distal end of an elongate member, and an inflator for selectively expanding the expandable member.

FIGS. 2A and 2B is a cross-sectional view of a subject's anatomy showing the device of FIG. 1 being introduced into the subject's vagina in a contracted condition (FIG. 2A) and expanded within the vagina for delivering light therapy (FIG. 2B).

### DETAILED DESCRIPTION OF THE EXEMPLARY EMBODIMENTS

Before the exemplary embodiments are described, it is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

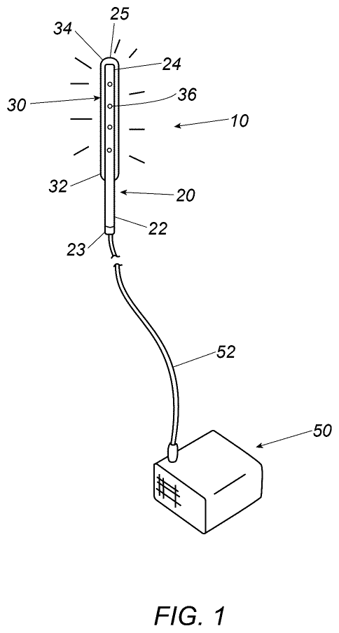

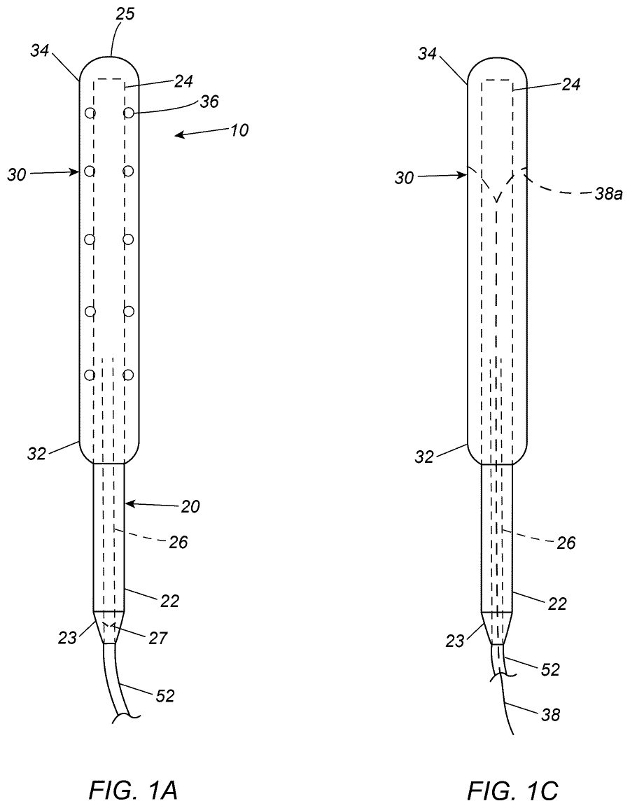

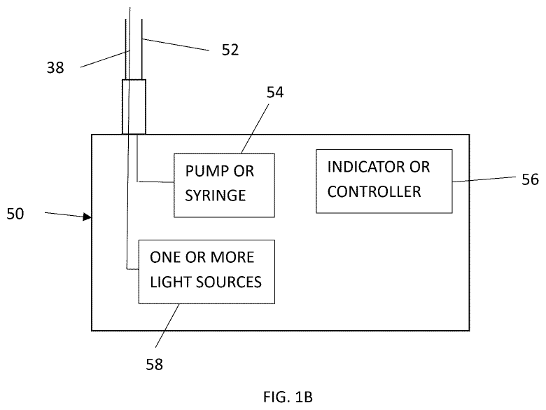

Turning to the drawings, FIG. 1 shows an exemplary embodiment of a vaginal therapy device 10 that includes a catheter, shaft, or other elongate member 20 including a proximal portion 22, a distal portion 24 carrying a balloon or other expandable member 30 and one or more light sources (not shown) for delivering light therapy to a subject, e.g., to treat bacterial or fungal infections, such as vaginitis, to treat pelvic inflammatory diseases, and/or to perform vaginal rejuvenation therapy, as described elsewhere herein.

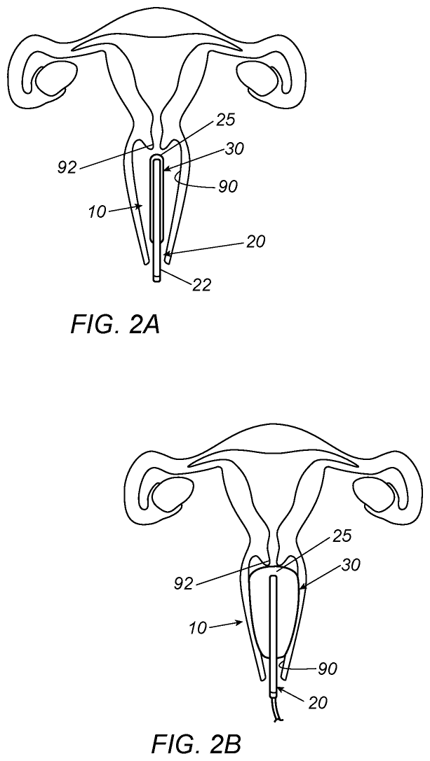

The distal portion 24 may be sized and/or shaped for insertion into a subject's vagina 90 with the balloon 30 in a contracted or delivery condition, e.g., as shown in FIG. 2A. In an exemplary embodiment, the distal portion 24 may be substantially rigid, semi-rigid, or flexible, while having sufficient column strength to insert the distal portion 24 into the subject's vagina 90, e.g., until a distal tip 25 of the elongate member 20 is disposed adjacent the subject's cervix 92. Optionally, the proximal portion 22 may also be substantially rigid, semi-rigid, or flexible, e.g., having sufficient column strength to advance the distal portion 24 into the subject's vagina while holding and/or manipulating the proximal portion 22. Optionally, the proximal portion 22 may include a handle (not shown), e.g., extending from and/or otherwise attached to the proximal portion 22 to facilitate manipulation of the device 10 during use.

The elongate member 20 may include one or more lumens, e.g., an inflation lumen (not shown) extending between the proximal and distal portions 22, 24 and communicating with an interior of the balloon 30 for delivering inflation media to expand the balloon 30 and/or for evacuating the inflation media to collapse the balloon 30. For example, as shown in FIG. 1, an inflator device 50 may be coupled to the proximal end 22 of the elongate member 20, e.g., via a hose or other line 52, for delivering inflation media into and/or evacuating inflation media from the balloon 30 via the inflation lumen. For example, the proximal portion 22 may include a port 23 communicating with the inflation lumen that includes a connector for removably connecting the hose 52 to the proximal portion 22. In addition, the elongate member 20 may include one or more additional lumens, e.g., carrying one or more wires, optical fibers, and the like (not shown) for delivering power or light to the one or more light sources on the distal portion 24. Alternatively, the inflator may be integrated into the shaft 20, e.g., within a handle on the proximal portion 22 (not shown).

In an exemplary embodiment, the device 10 may include a rigid or flexible “wand” carrying an uninflated, stretchable balloon 30 covering a desired length along the distal portion 24, e.g., as shown in FIGS. 1 and 2A. For example, as shown in FIG. 1, the balloon 30 may include a proximal end 32 attached to the shaft 20, and an enclosed distal end 34 that extends over the distal tip 25. Alternatively, the balloon may include an annular distal end (not shown) attached to the distal portion 24, e.g., adjacent the distal tip 25. FIG. 2B view shows the balloon 30 inflated, e.g., with air or other fluid, as described further elsewhere herein. One or more LEDs or other light sources (not shown) may be mounted in the wall and/or otherwise positioned or carried on the distal portion 10 of the shaft 20 over the length covered by the balloon 30. Once the distal portion 24 is inserted and the balloon 30 is inflated, the light source(s) may be turned on to irradiate the vaginal wall through the inflated balloon 30, e.g., similar to the devices disclosed in U.S. Pat. No. 10,004,918 and U.S. Publication No. 2018/0361170, the entire disclosures of which are expressly incorporated by reference herein.

In this configuration, for light to travel effectively from the distal portion 24 to the vaginal wall, the balloon 30 may be (i) thin walled and made of a material to allow light to pass without significant attenuation, e.g., material that is transparent to at least the wavelength(s) of light emitted by the light source, and (ii) inflated with a low-attenuation material such as a compressible fluid, such as air or carbon dioxide. Alternatively, one or more light sources may be mounted, embedded in, or otherwise fixed to the wall of the balloon 30. For example, a plurality of LEDs, and the like (not shown) may be attached to the wall of the balloon 30 that are spaced apart from one another to transmit light radially outwardly from the balloon 30.

Alternatively, a plurality of light fibers (not shown) may be embedded in or otherwise attached to the wall of the balloon 30. In this alternative, one or more light sources (also not shown) may be provided on the proximal portion 22 and/or on the inflator device 50 that may communicate light to the light fibers, e.g., via fibers within a lumen of the shaft 20 (not shown), with ends of the fibers arranged on the balloon 30 transmitting the light radially outwardly from the balloon 30.

Optionally, because the operator cannot see the balloon 30 once inserted into the vagina 90, an indicator or controller (not shown) may be provided to inform the operator of the status of the balloon 30, e.g., based on the inflating volume and/or pressure. For example, the inflator device 50 may include a manual or motorized pump, a syringe, and the like (not shown), that may be coupled to a valve, e.g., on the connector 23 on the proximal portion 22 of the shaft 20.

The inflator device 50 may be configured to deliver inflation media through the inflation lumen of the shaft 20 into the interior of the balloon 30, e.g., to deliver a predetermined volume of inflation media to inflate the balloon 30 to a desired size. In addition or alternatively, the inflator device 50 may monitor the pressure of the inflation media being delivered, e.g., to ensure a maximum pressure is not exceeded. In addition or alternatively, one or more actuators (not shown) may be provided on the proximal portion 22, e.g., on a handle attached to the proximal portion 22, which may be activated by the user to activate/deactivate the inflator device 50 and/or turn the light source(s) on and off, as desired.

For example, a compressible gas or other fluid may be delivered via the inflator device 50 to expand the balloon 30 and dilate and/or otherwise shape the subject's anatomy surrounding the vagina 90 before light delivery, e.g., to dilate the vagina and/or otherwise expose tissue surfaces for treatment. The compressible fluid and elastic balloon may also allow the balloon 30 to conform at least somewhat to the user's anatomy, e.g., to minimize gaps between the balloon and vaginal wall. Exemplary gases may include carbon dioxide or other biocompatible gas (particularly for applications where rupture of the balloon 30 may expose the fluid to the subject's vascular system). Assuming use of an ideal gas, the inflated volume may be inversely proportional to the pressure to which the balloon 30 is inflated. Since the amount of pressure to achieve a desired volume may vary significantly from one woman's vagina to another, depending on such factors as body weight, pelvic floor dimensions, muscle tone, number of prior pregnancies, etc., inflating the balloon 30 to a predetermined volume may ensure proper inflation independent of the anatomy encountered. A manual inflator device may include an actuator and visual indicia (not shown) to identify the volume being delivered, or a motorized inflator device may deliver fluid until the predetermined volume is delivered and then automatically shut off. In addition or alternatively, external imaging, e.g., ultrasound, may be used to monitor inflation of the balloon 30 to confirm when a desired size and/or shape are attained.

Alternatively, multiple valves may be provided within the shaft 20 that are coupled in series such that, once desired pressure, e.g., five psi is achieved, the inflator device 50 may automatically shut off. In addition or alternatively, the device 10 may include a relief valve (not shown) to release excess pressure and/or prevent over-inflation of the balloon 30 during use.

In another embodiment, the balloon 30 may be made of an inelastic material to provide a balloon with a single volume and/or shape once inflated (i.e., the balloon does not expand appreciably with increased pressure once fully inflated). For example, the balloon 30 may have a desired shape configured to dilate and/or otherwise shape the vaginal wall, e.g., to enhance exposure to the light transmitted by the device. The material may be reasonably strong with low light attenuation, such as cellophane, for example.

In this embodiment, the balloon 30 may be inflated to a pressure that provides full expansion of the balloon 30 within any typical vaginal cavity, e.g., using a compressible fluid, such as carbon dioxide, or an incompressible fluid, that is non-attenuating to the light transmitted by the light source(s). In this alternative, no indicator to inform the operator of the balloon volume may be required if incompressible fluid is used given the fixed shape of the balloon. As in the earlier embodiment, one or more LEDs or other light sources may be activated to emit light from the shaft 20 through the and balloon wall to the vaginal wall. Examples of compressible fluid may include carbon dioxide, nitrogen, and the like, e.g., in case the balloon ruptures and the gas enters the blood stream.

In another embodiment, the stiff balloon material may be a thicker, more-robust material that substantially attenuates light. In such a configuration, one or more LEDs or other light sources may be mounted on or in the balloon wall. In this configuration, an incompressible fluid, such as water, may be used to inflate the balloon.

The devices described herein may be used by an individual on their own, e.g., at home or other non-medical setting. Alternatively, the devices may be operated by a medical professional, e.g., in a doctor's office, hospital, or other location, to provide one or more treatments delivering vaginal light therapy of a patient.

For example, initially, as shown in FIG. 2A, the distal portion 24 of the device 10 (carrying the balloon 30 and one or more light sources) may be inserted into a vaginal canal 90 with the balloon 30 in a contracted condition. Once positioned as desired, e.g., with the distal tip 25 against or immediately adjacent the cervix 92, the balloon 30 may be expanded, e.g., by connecting the hose 52 of the inflator device to the proximal portion 22 and delivering inflation media, e.g., a predetermined volume of biocompatible fluid. Optionally, particularly if the balloon 30 is formed from elastic material, positioning and/or inflation of the balloon 30 may be monitored by the operator, e.g., using ultrasound or other external imaging, e.g., to ensure the balloon 30 is inflated to a desired size and/or shape. The one or more light sources may then be activated to emit light outwardly at one or more wavelengths, e.g., within a range of non-UV or other suitable germicidal light. Upon completion of a desired treatment, the balloon 30 may be deflated, and the device 10 removed from the vaginal canal 90 after treatment. The device 10 may sterilized and/or otherwise cleaned for subsequent reuse, or may be discarded as a single-use device.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for vaginal light therapy of a patient, comprising:
an elongate member comprising a proximal portion, a distal portion sized for introduction into a vaginal canal terminating in a distal tip, and one or more light sources on the distal portion;
an expandable member carried on the distal portion and surrounding the one or more light sources, the expandable member comprising a proximal end attached to the elongate member proximal to the distal tip and an enclosed distal end that extends over the distal tip of the distal portion, the expandable member configured to emit light outwardly from the one or more light sources at one or more wavelengths within a range of germicidal light to treat one or more pathogens without injuring vaginal tissue; and
an inflator for expanding the expandable member from a delivery condition for introduction into a vaginal canal, and an expandable condition once inserted for delivering the light to treat one or more conditions,
wherein the one or more light sources comprise a plurality of light sources spaced apart from one another on a wall of the distal portion to transmit light radially outwardly through the expandable member.

2. The device of claim 1, wherein the inflator is connectable to the proximal portion of the elongate member and the elongate member includes an inflation lumen extending between the proximal and distal portions for delivering inflation media into an interior of the expandable member.

3. The device of claim 1, wherein the inflator is mounted on or in the elongate member and a valve is provided in the proximal portion for delivering inflation media via the inflator into an interior of the expandable member.

4. The device of claim 1, wherein the inflator comprises a manual or motorized pump.

5. The device of claim 1, wherein the inflator comprises a syringe filled with a predetermined volume of inflation media.

6. The device of claim 1, wherein the expandable member comprises a balloon formed from elastic material such that the balloon at least partially conforms to the subject's anatomy when expanded.

7. The device of claim 1, wherein the expandable member comprises a balloon formed from elastic material configured to dilate the subject's anatomy when expanded.

8. The device of claim 1, wherein the plurality of light sources comprise a plurality of LEDs mounted to the wall and spaced apart from one another on the distal portion.

9. A device for vaginal light therapy of a patient, comprising:
an elongate member comprising a proximal portion, and a distal portion sized for introduction into a vaginal canal terminating in a distal tip;
an expandable member carried on the distal portion carrying a plurality of light sources configured to emit light radially outwardly at one or more wavelengths within a range of germicidal light to treat one or more pathogens without injuring vaginal tissue, the expandable member comprising a proximal end attached to the elongate member and an enclosed distal end that extends over the distal tip of the distal portion; and
an inflator for expanding the expandable member from a delivery condition for introduction into a vaginal canal, and an expandable condition once inserted for delivering the light to treat one or more conditions.

10. The device of claim 9, wherein the inflator is connectable to the proximal portion of the elongate member and the elongate member includes an inflation lumen extending between the proximal and distal portions for delivering inflation media into an interior of the expandable member.

11. The device of claim 9, wherein the inflator is mounted on or in the elongate member and a valve is provided in the proximal portion for delivering inflation media via the inflator into an interior of the expandable member.

12. The device of claim 9, wherein the inflator comprises a manual or motorized pump.

13. The device of claim 9, wherein the inflator comprises a syringe filled with a predetermined volume of inflation media.

14. The device of claim 9, wherein the expandable member comprises a balloon formed from elastic material such that the balloon at least partially conforms to the subject's anatomy when expanded.

15. The device of claim 9, wherein the expandable member comprises a balloon formed from elastic material configured to dilate the subject's anatomy when expanded.

16. The device of claim 9, wherein the light sources comprise a plurality of LEDs spaced apart from one another on a wall of the expandable member to transmit light radially outwardly from the expandable member.

17. The device of claim 9, wherein the light sources comprises a plurality of optic fibers with ends spaced apart from one another and a light coupled to the optic fibers to deliver light through the fibers and out the ends radially outwardly from the expandable member.

18. The device of claim 17, wherein the light is provided in the inflator, and wherein the elongate member includes one or more source optic fibers communicating with the light and the optic fibers on the wall of the expandable member.

19. A method for vaginal light therapy of a patient, comprising:
providing a treatment device comprising an elongate member including a distal portion carrying an expandable member comprising a proximal end attached to the elongate member and an enclosed distal end that extends over a distal tip of the distal portion, the treatment devices comprising one or more light sources;
inserting the distal portion into a vaginal canal with the expandable member in a contracted condition;
expanding the expandable member within the vaginal canal;
activating the one or more light sources to emit light radially outwardly along the distal portion from the expandable member at one or more wavelengths within a range of germicidal light to treat one or more pathogens without injuring vaginal tissue; and
removing the device from the vaginal canal after treatment.

20. The method of claim 19, wherein expansion of the expandable member is monitored using external imaging.

21. The method of claim 19, wherein the expandable member comprises an elastic balloon configured to conform to the patient's anatomy to minimize gaps between the balloon and a wall of the vaginal canal.

22. The method of claim 19, wherein the expandable member covers sufficient length along the distal portion to irradiate the length of the vaginal canal when the expandable member is expanded.

23. The method of claim 19, wherein the distal portion is inserted into the vaginal canal such that a distal tip of the expandable member is positioned adjacent the cervix.

24. The method of claim 19, wherein the one or more light sources comprise a plurality of light sources spaced apart from one another on a wall of the distal portion to transmit light radially outwardly through the expandable member.
